(19) United States
(12) Patent Application Publication
(10) Pub. No.: US 2021/0253567 A1
(43) Pub. Date: Aug. 19, 2021

(54) AMORPHOUS AND CRYSTALLINE FORMS OF VALBENAZINE SALT

(71) Applicant: SUZHOU PENGXU PHARMATECH CO., LTD., Suzhou, JiangSu (CN)

(72) Inventors: Peng WANG, New York, NY (US); Pixu LI, Wujiang, Suzhou (CN); Qiang WEI, Wujiang, Suzhou (CN); Dingquan LIU, Wujiang, Suzhou (CN); Hao WU, Wujiang, Suzhou (CN); He CHEN, Wujiang, Suzhou (CN); Wenhui YAO, Wujiang, Suzhou (CN)

(21) Appl. No.: 17/253,498

(22) PCT Filed: Aug. 13, 2019
(86) PCT No.: PCT/US2019/046358

§ 371 Date: Dec. 17, 2020

(30) Foreign Application Priority Data
Aug. 14, 2018 (CN) 201811104129.1

(51) Int. Cl. C07D 455/06

(52) U.S. Cl. CPC C07D 455/06; C07B 2200/13

(57) ABSTRACT

The present invention relates to amorphous and crystalline forms of valbenazine salt and the methods for their preparation.

### CROSS REFERENCE TO RELATED APPLICATION

This application claims the benefit of CN patent No. 201810925349.4 filed on Aug. 14, 2018; the disclosure of which is incorporated herein by reference in its entirety.

### FIELD OF THE INVENTION

The present application relates to amorphous and crystalline forms of valbenazine salt and the methods for their preparation.

### BACKGROUND OF THE INVENTION

Valbenazine (INGREZZA), a vesicular monoamine transporter 2 (VMAT2) inhibitor, with the chemical name, L-Valine, (2R, 3R, 11bR)-1, 3, 4, 6, 7, 11b-hexahydro-9, 10-dimethoxy-3-(2-methylpropyl)-2H-benzo[a]quinolizin-2-yl ester, was approved by FDA on Apr. 11, 2017. Valbenazine has a structure of formula (I), herein after named Compound I. The mechanism of action of valbenazine in the treatment of tardive dyskinesia is unknown, but is thought to be mediated through the reversible inhibition of vesicular monoamine transporter 2 (VMAT2), a transporter that regulates monoamine uptake from the cytoplasm to the synaptic vesicle for storage and release.

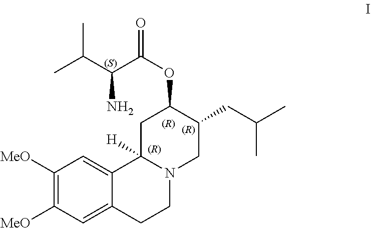

International Patent Application Publication No. WO2017075340 describes crystalline Forms I, II, III, IV and amorphous of valbenazine tosylate, crystalline Forms I, II and amorphous of valbenazine hydrochloride, and the processes for the preparation of the polymorphic forms.

Polymorphic crystalline phases, as well as salt forms, of APIs represents an option to design materials with improved processing properties (handling and workability), better storage stability, and can provide a procedure to increase the purity of API. New polymorphs or salts are also useful as desirable intermediate phases to drive the conversion into the polymorph of interest. Novel crystalline forms of APIs may offer better processing and physicochemical properties, such as bioavailability, stability, process ability, and purification ability. Some novel salt forms may serve as intermediate crystal forms to get high purity APIs or to reduce the level of genotoxic impurities in the final product.

The stability and purification ability of valbenazine tosylate and valbenazine hydrochloride were found to be limited. In addition, the presence of p-toluenesulfonic acid and alcohol increases the risk of genotoxic impurity formation. To date, crystalline valbenazine tosylate is the only form which is used for drug formulation, and it is also used for scaled up production of active pharmaceutical ingredients (API). In addition to above-described salts, no other salt is disclosed. Therefore, it is significant to develop a novel salt of valbenazine with better stability, better purification capability, higher chiral isomer purity and no risk of genotoxic impurity formation for drug development. With lots of experiments being carried out, inventors of present application finally found crystalline Form A1, Form A2, Form A3, Form A4 and Form A5 of valbenazine oxalate, amorphous of valbenazine hydrobromide, crystalline Form B of valbenazine L-tartrate, crystalline Form C of valbenazine Di-p-toluoyl-L-tartrate, crystalline Form D of valbenazine D-tartrate, crystalline Form X of valbenazine, which are beneficial for the process development and the production of API. The crystalline form A1 of valbenazine oxalate has advantages of good stability, simple preparation process and good purification capability. The crystalline form A1 of valbenazine oxalate provides a new and better choice for the development of valbenazine drug product.

### SUMMARY

The present application relates to novel polymorphic forms of valbenazine and its salt, and the processes for their preparation.

In particular embodiments, the present invention relates to crystalline Form A1, Form A2, Form A3, Form A4 and Form A5 of valbenazine oxalate, amorphous of valbenazine hydrobromide, crystalline Form B of valbenazine L-tartrate, crystalline Form C of valbenazine Di-p-toluoyl-L-tartrate, crystalline Form D of valbenazine D-tartrate, crystalline Form X of valbenazine, characterized by X-ray powder diffraction (“XRPD”), Differential Scanning calorimetry (“DSC”), and the processes for their preparation.

The present invention further provides crystallization processes for preparing crystalline valbenazine or its salt. The valbenazine starting material can be produced by any suitable method, including synthesis methods known in the art.

The present invention provides crystalline Form A1 of valbenazine oxalate characterized by a XRPD pattern depicted in FIG. 1 comprising peaks at 2-theta angles of about 5.4°±0.2°, 7.1°±0.2°, 13.4°±0.2°.

The present invention provides crystalline Form A2 of valbenazine oxalate characterized by a XRPD pattern depicted in FIG. 11 comprising peaks at 2-theta angles of about 15.1°±0.2°, 20.2°±0.2°, 20.6°±0.2°.

The present invention provides crystalline Form A3 of valbenazine oxalate characterized by a XRPD pattern depicted in FIG. 12 comprising peaks at 2-theta angles of about 6.7°±0.2°, 7.1°±0.2°, 9.5°±0.2°.

The present invention provides crystalline Form A4 of valbenazine oxalate characterized by a XRPD pattern depicted in FIG. 13 comprising peaks at 2-theta angles of about 5.5°±0.2°, 8.8°±0.2°, 12.7°±0.2°.

The present invention provides crystalline Form A5 of valbenazine oxalate characterized by a XRPD pattern depicted in FIG. 14 comprising peaks at 2-theta angles of about 3.4°±0.2°, 6.1°±0.2°, 7.0°±0.2°.

The present invention provides amorphous of valbenazine hydrobromide characterized by a XRPD pattern depicted in FIG. 2.

The present invention provides crystalline Form B of valbenazine L-tartrate characterized by a XRPD pattern depicted in FIG. 3 comprising peaks at 2-theta angles of about 9.8°±0.2°, 11.0°±0.2°, 14.2°±0.2°.

The present invention provides crystalline Form C of valbenazine Di-p-toluoyl-L-tartrate characterized by a XRPD pattern depicted in FIG. 4 comprising peaks at 2-theta angles of about 5.4°±0.2°, 6.6°±0.2°, 14.0°±0.2°.

The present invention provides crystalline Form D of valbenazine D-tartrate characterized by a XRPD pattern depicted in FIG. 5 comprising peaks at 2-theta angles of about 6.7°±0.2°, 7.8°±0.2°, 18.2°±0.2°.

The present invention provides crystalline Form X of valbenazine characterized by a XRPD pattern depicted in FIG. 19 comprising peaks at 2-theta angles of about 5.9°±0.2°, 6.7°±0.2°, 9.8°±0.2°, 18.0°±0.2°.

### BRIEF DESCRIPTION OF THE DRAWINGS

FIG. 1. Exemplary XRPD pattern of crystalline Form A1 of valbenazine oxalate.

FIG. 2. Exemplary XRPD pattern of amorphous of valbenazine hydrobromide.

FIG. 3. Exemplary XRPD pattern of crystalline Form B of valbenazine L-tartrate.

FIG. 4. Exemplary XRPD pattern of crystalline Form C of valbenazine Di-p-toluoyl-L-tartrate.

FIG. 5. Exemplary XRPD pattern of crystalline Form D of valbenazine D-tartrate.

FIG. 6. Exemplary DSC curve of crystalline Form A1 of valbenazine oxalate.

FIG. 7. Exemplary DSC curve of amorphous of valbenazine hydrobromide.

FIG. 8. Exemplary DSC curve of crystalline Form B of valbenazine L-tartrate.

FIG. 9. Exemplary DSC curve of crystalline Form C of valbenazine Di-p-toluoyl-L-tartrate.

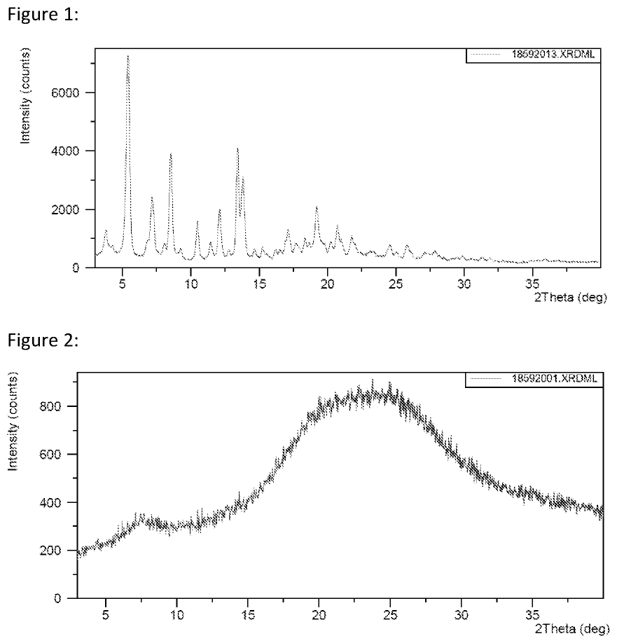

FIG. 1.0. Exemplary DSC curve of crystalline Form D of valbenazine D-tartrate.

FIG. 11. Exemplary XRPD pattern of crystalline Form A2 of valbenazine oxalate.

FIG. 12, Exemplary XRPD pattern of crystalline Form A3 of valbenazine oxalate.

FIG. 13. Exemplary XRPD pattern of crystalline Form A4 of valbenazine oxalate.

FIG. 14. Exemplary XRPD pattern of crystalline Form A5 of valbenazine oxalate.

FIG. 15. Exemplary DSC curve of crystalline Form A2 of valbenazine oxalate.

FIG. 16, Exemplary DSC curve of crystalline Form A3 of valbenazine oxalate.

FIG. 17. Exemplary DSC curve of crystalline Form A4 of valbenazine oxalate.

FIG. 18. Exemplary DSC curve of crystalline Form A5 of valbenazine oxalate.

FIG. 19. Exemplary XRPD pattern of crystalline Form X of valbenazine.

FIG. 20, Exemplary DSC curve of crystalline Form X of valbenazine.

### DETAILED DESCRIPTION

One aspect of the present application relates to crystalline forms of valbenazine oxalate designated herein as Form A1, Form A2, Form A3, Form A4 and Form A5 and processes for preparation thereof.

Crystalline Form A1, Form A2, Form A3, Form A4 and Form A5 of valbenazine oxalate may be characterized by any one or more analytical techniques, which include XRPD patterns and differential scanning calorimetry (DSC) curves.

In an embodiment, the present invention provides crystalline Form A1 of valbenazine oxalate characterized by a XRPD pattern depicted in FIG. 1 comprising peaks at 2-theta angles of about 5.4°±0.2°, 7.1°±0.2°, 13.4°±0.2°.

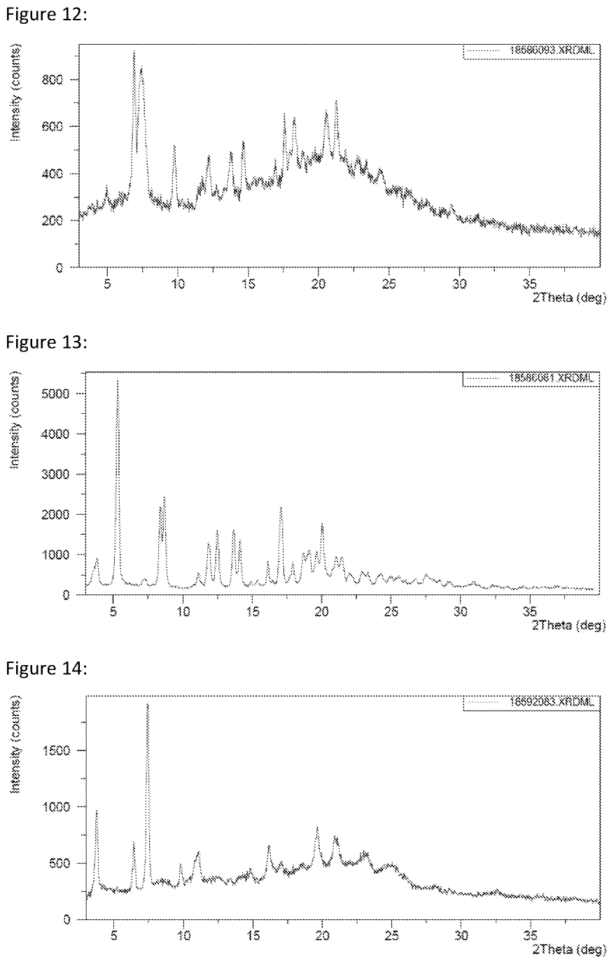

In another aspect, crystalline Form A1 of valbenazine oxalate was characterized by a DSC profile in accordance with the profile shown in FIG. 6 in an embodiment, the present invention provides crystalline Form A2 of valbenazine oxalate characterized by a XRPD pattern depicted in FIG. 11 comprising peaks at 2-theta angles of about, 15.1°±0.2°, 20.2°±0.2°, 20.6°±0.2°.

In another aspect, crystalline Form A2 of valbenazine oxalate was characterized by a DSC profile in accordance with the profile shown in FIG. 15

In an embodiment, the present invention provides crystalline Form A3 of valbenazine oxalate characterized by a XRPD pattern depicted in FIG. 12 comprising peaks at 2-theta angles of about 6.7°±0.2°, 7.1°±0.2°, 9.5°±0.2°.

In another aspect, crystalline Form A3 of valbenazine oxalate was characterized by a DSC profile in accordance with the profile shown in FIG. 16

In an embodiment, the present invention provides crystalline Form A4 of valbenazine oxalate characterized by a XRPD pattern depicted in FIG. 13 comprising peaks at 2-theta angles of about 5.5°±0.2°, 8.8°±0.2°, 12.7°±0.2°.

In another aspect, crystalline Form A4 of valbenazine oxalate was characterized by a DSC profile in accordance with the profile shown in FIG. 17

In an embodiment, the present invention provides crystalline Form A5 of valbenazine oxalate characterized by a XRPD pattern depicted in FIG. 14 comprising peaks at 2-theta angles of about 3.4°±0.2′, 6.1°±0.2°, 7.0°±0.2°.

In another aspect, crystalline Form A5 of valbenazine oxalate was characterized by a DSC profile in accordance with the profile shown in FIG. 18

The other aspect of the present application relates to amorphous of valbenazine hydrobromide and the processes for preparation thereof.

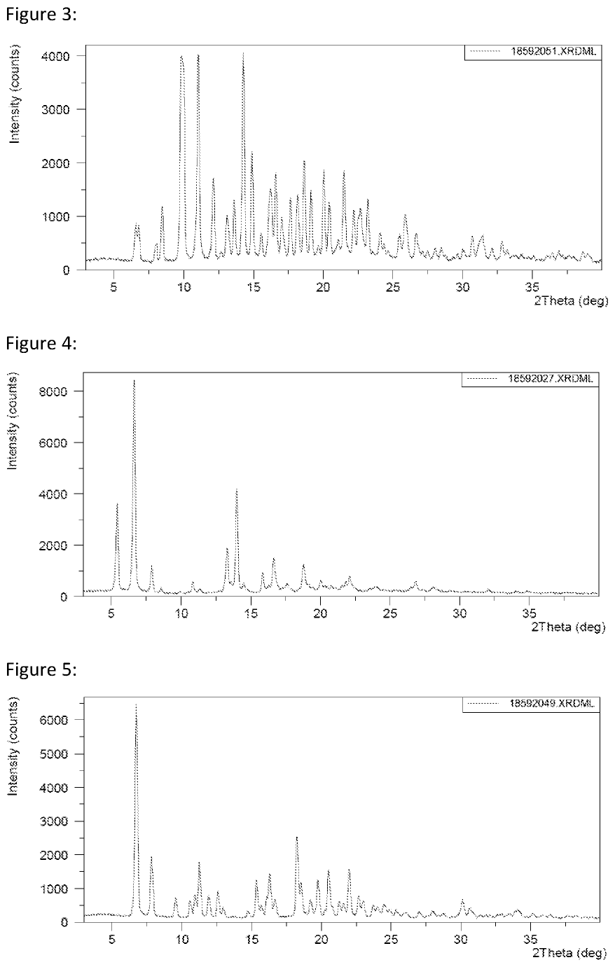

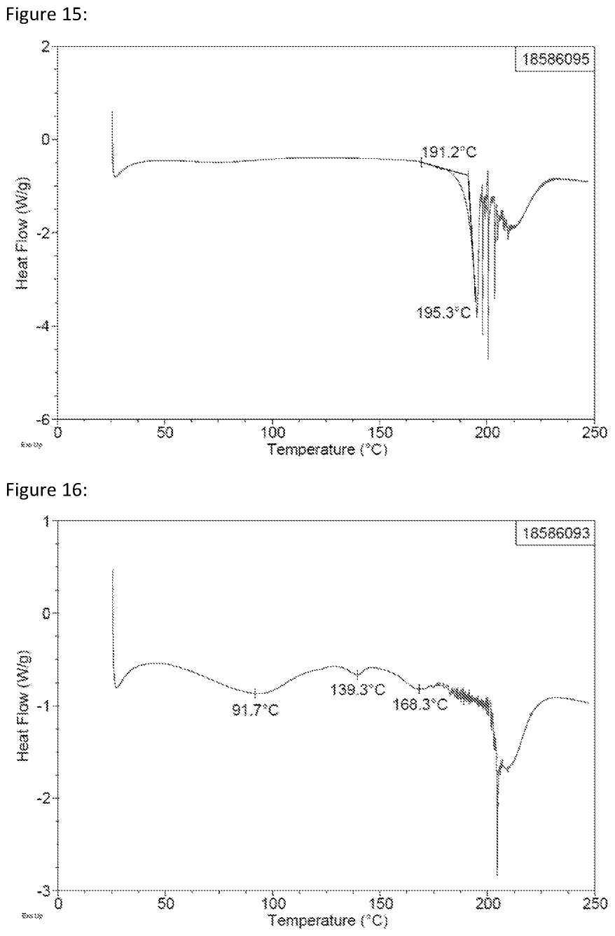

Amorphous of valbenazine hydrobromide may be characterized by XRPD patterns depicted in FIG. 2, and DSC in FIG. 7

The other aspect of the present application relates to crystalline forms of valbenazine L-tartrate designated herein as Form B and the processes for preparation thereof.

Crystalline Form B of valbenazine L-tartrate may be characterized by any one or more analytical techniques, which include XRPD patterns and differential scanning calorimetry (DSC) curves.

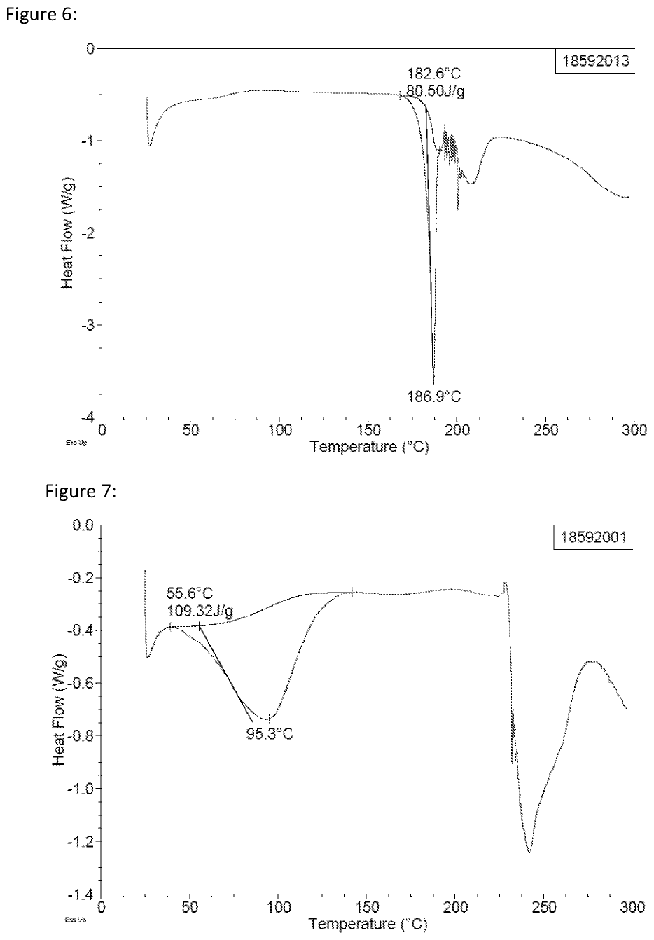

In an embodiment, the present invention provides crystalline Form B of valbenazine L-tartrate characterized by a XRPD pattern depicted in FIG. 3 comprising peaks at 2-theta angles of about 9.8°±0.2°, 11.0°±0.2°, 14.2°±0.2°.

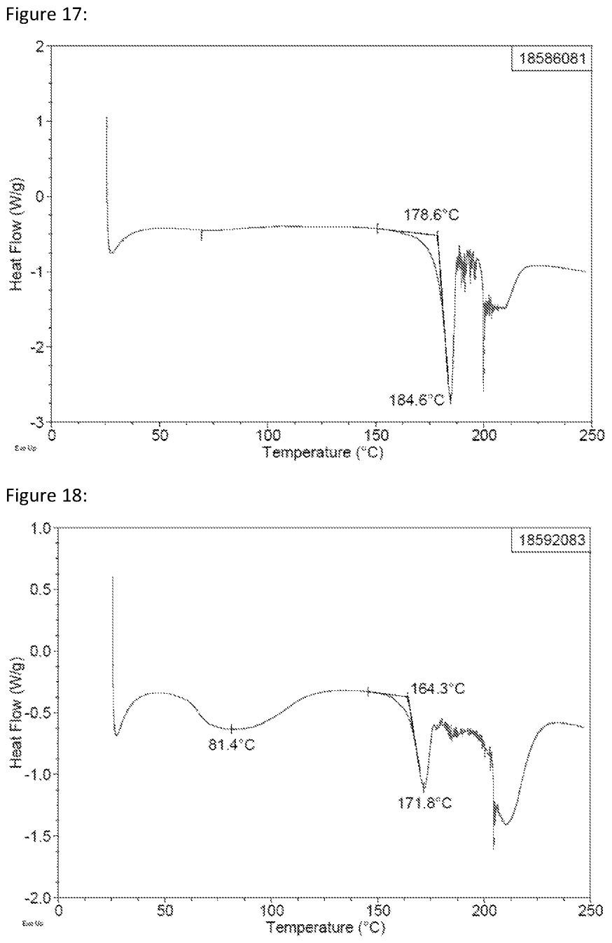

In another aspect, crystalline Form B of valbenazine L-tartrate was characterized by a DSC profile in accordance with the profile shown in FIG. 8

The other aspect of the present application relates to crystalline form of valbenazine Di-p-toluoyl-L-tartrate designated herein as Form C and the processes for the preparation thereof.

Crystalline Form C of valbenazine Di-p-toluoyl-L-tartrate may be characterized by any one or more analytical techniques, which include XRPD patterns and differential scanning calorimetry (DSC) curves.

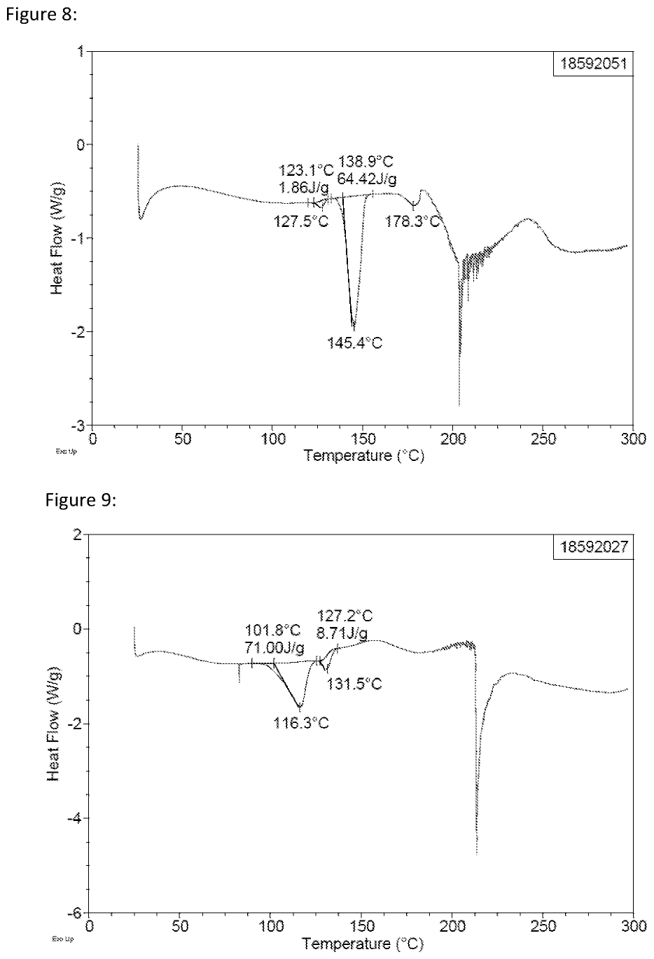

In an embodiment, the present invention provides crystalline Form C of valbenazine Di-p-toluoyl-L-tartrate characterized by a XRPD pattern depicted in FIG. 4 comprising peaks at 2-theta angles of about 5.4°±0.2°, 6.6°±0.2°, 14.0°±0.2°.

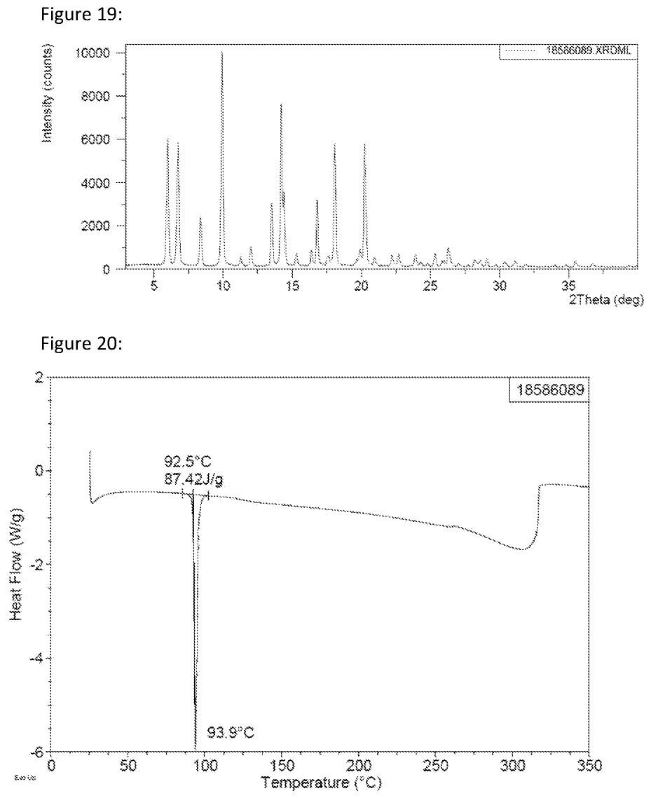

In another aspect, crystalline Form C of valbenazine Di-p-toluoyl-L-tartrate was characterized by a DSC profile in accordance with the profile shown in FIG. 9

The other aspect of the present application relates to crystalline form of valbenazine D-tartrate designated herein as Form D and processes for preparation thereof.

Crystalline Form D of valbenazine D-tartrate may be characterized by any one or more analytical techniques, which include XRPD patterns and differential scanning calorimetry (DSC) curves.

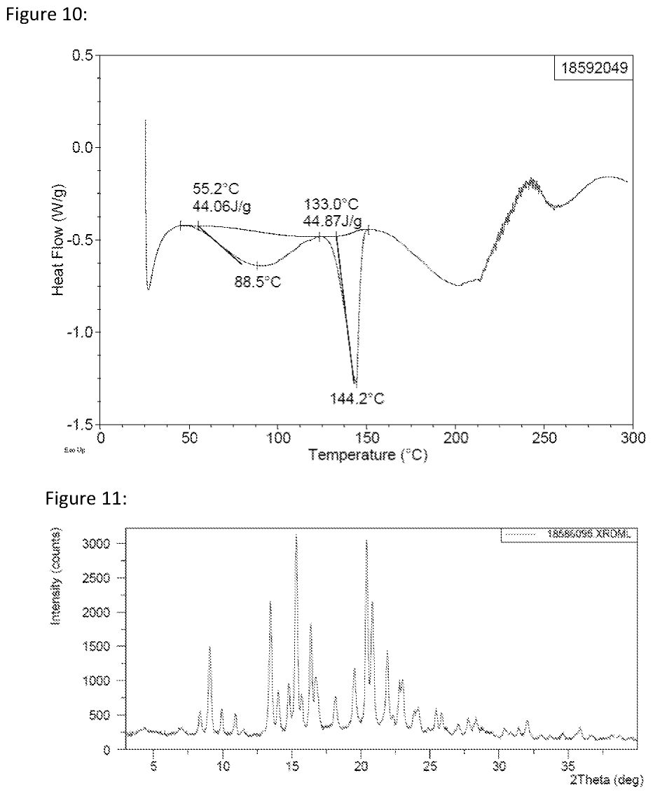

In an embodiment, the present invention provides crystalline Form D of valbenazine D-tartrate characterized by a XRPD pattern depicted in FIG. 5 comprising peaks at 2-theta angles of about 6.7°±0.2°, 7.8°±0.2°, 18.2°±0.2°.

In another aspect, crystalline Form D of valbenazine D-tartrate was characterized by a DSC profile in accordance with the profile shown in FIG. 10

The other one aspect of the present application relates to crystalline form of valbenazine designated herein as Form X and processes for the preparation thereof.

Crystalline Form X of valbenazine may be characterized by any one or more analytical techniques, which include XRPD patterns and differential scanning calorimetry (DSC) curves.

In an embodiment, the present invention provides crystalline Form X of valbenazine characterized by a XRPD pattern depicted in FIG. 19 comprising peaks at 2-theta angles of about 5.9°±0.2°, 6.7°±0.2°, 9.8°±0.2°, 18.0°±0.2°.

In another aspect, crystalline Form X of valbenazine is characterized by a DSC profile in accordance with the profile shown in FIG. 20

The crystalline phases, isolated by the methods of the present application can be analyzed by Powder X-ray Diffraction (XRPD) was performed on a PANalytical Empyrean X-ray Powder Diffractometer, equipped with a Cu-anode (λ=1.54 A), X-ray source operated at 45 kV, 40 mA, and Start Position [°2Th.]: 3.0056; End Position [°2Th.]: 39.9906; Step Size [°2Th.]: 0.0167; Scan Step Time [s]: 17.8500; K-Alpha1 [Å]: 1.54060; K-Alpha2 [Å]: 1.54443.

The DSC profiles were registered using a TA200 DSC instrument. The sample was weighed in an aluminum pan sealed with a pierced aluminum cover. The analysis was performed heating the sample from 25° C. to 300° C.

### EXAMPLES

### Example 1

### Preparation of Compound I Oxalate Crystalline Form A1

A solution of compound I (6.06 g, purity: 97.5%) in DCM (120 mL) was concentrated to about 15 mL under vacuum. After solvent exchange to isopropyl acetate (IPAc), the IPAc solution was heated to 40-50° C., followed by drop-wise addition of a solution of anhydrous oxalic acid (1.0 eq) in IPAc (4 volume) at 40-50° C. After being stirred and held for 2-3 h at 40-50° C., the mixture was cooled to 20-30° C. and stirred for 1-2 h. The resulting suspension was filtered. The cake was washed with IPAc, and dried to afford valbenazine oxalate as an off-white solid. (6.63 g, purity: 99.4%, yield: 90%). ¹H-NMR (DMSO-d₆, 400 MHz) δ: 8.04 (4H, brs, active hydrogen), 6.88 (2H, 2s, ArH), 4.76 (1H, td, J=9.8 Hz, 4.8 Hz), 3.78 (1H, d, J=6.4 Hz), 3.71 (6H, —OCH₃), 3.37 (1H, m), 3.11 (1H, m), 3.05 (1H, m), 2.91 (1H, m), 2.59 (2H, m), 2.48 (1H, m), 2.17 (2H, m), 1.89 (1H, m), 1.63 (1H, m), 1.46 (1H, m), 1.27 (1H, m), 1.03 (1H, m), 0.98 (6H, dd, J=6.8 Hz, 9.2 Hz, -iPr), 0.87 (6H, dd, J=5.6 Hz, 11.6 Hz, -iPr). The XRPD pattern of crystalline form A1 was shown in table 1.

TABLE 1

| 2theta | d spacing | Intensity % |
|---|---|---|
| 5.38 | 16.43 | 100.00 |
| 7.14 | 12.38 | 31.66 |
| 8.50 | 10.40 | 50.93 |
| 10.50 | 8.43 | 18.54 |
| 12.10 | 7.31 | 24.98 |
| 13.41 | 6.60 | 56.19 |
| 13.81 | 6.41 | 40.02 |
| 17.08 | 5.19 | 15.47 |
| 19.20 | 4.62 | 26.81 |
| 20.71 | 4.29 | 17.72 |

### Example 2

### Preparation of Compound I Oxalate Crystalline Form A2

A solid of compound I (1.5 g, purity: 98.8%) was added IPAc (30 mL), follow by addition of anhydrous oxalic acid 322 mg (1 eq). The mixture was heated to 50-60° C. and stirred for about 20-30 min, cooled to 20-30° C. and stirred for 16 h. The resulting suspension was filtered. The cake was washed with IPAc, and dried to afford valbenazine oxalate as an off-white solid. (780 mg, purity: 99.7%, yield: 43%). The XRPD pattern of crystalline form A2 was shown in table 2.

TABLE 2

| 2theta | d spacing | Intensity % |
|---|---|---|
| 8.82 | 10.02 | 43.51 |
| 13.19 | 6.71 | 65.15 |
| 13.77 | 6.43 | 23.03 |
| 15.52 | 6.10 | 26.71 |
| 15.07 | 5.88 | 100.00 |
| 16.12 | 5.50 | 55.53 |
| 16.46 | 5.39 | 28.97 |
| 19.32 | 4.60 | 33.21 |
| 20.16 | 4.41 | 97.45 |
| 20.58 | 4.32 | 67.86 |
| 21.68 | 4.10 | 43.42 |
| 22.54 | 3.94 | 28.95 |
| 22.80 | 3.90 | 29.09 |

### Example 3

### Preparation of Compound I Oxalate Crystalline Form A3

A solution of compound I (2.57 g, purity: 97.5%) in DCM (60 mL) was mixed with anhydrous oxalic acid (1.0 eq). The mixture was heated to reflux for 2 h. The mixture was cooled to 20-30° C. and stirred for 16-20 h. The resulting suspension was filtered. The cake was washed with DCM, and dried to afford valbenazine oxalate as an off-white solid. (2.87 g, purity: 99.4%, yield: 92%). The XRPD pattern of crystalline form A3 was shown in table 3.

TABLE 3

| 2theta | d spacing | Intensity % |
|---|---|---|
| 4.73 | 18.70 | 11.66 |
| 6.69 | 13.22 | 100.00 |
| 7.06 | 12.51 | 48.60 |
| 9.50 | 9.31 | 48.07 |
| 13.61 | 6.51 | 33.75 |
| 14.41 | 6.14 | 45.45 |
| 16.75 | 5.29 | 16.64 |
| 17.40 | 5.10 | 37.07 |
| 17.95 | 4.94 | 23.89 |
| 18.65 | 4.76 | 16.76 |
| 19.35 | 4.59 | 13.80 |
| 20.30 | 4.38 | 26.51 |
| 21.02 | 4.23 | 35.80 |
| 21.64 | 4.11 | 15.98 |
| 22.66 | 3.92 | 19.04 |
| 23.16 | 3.84 | 11.47 |
| 24.26 | 3.67 | 19.11 |
| 29.19 | 3.06 | 11.89 |

### Example 4

### Preparation of Compound I Oxalate Crystalline Form A3

A solid of compound I (1.5 g, purity: 98.8%) was added DCM (30 mL), follow by addition of anhydrous oxalic acid 322 mg (1 eq). After being stirred and held for 4 h at 20-30° C., the resulting suspension was filtered. The cake was washed with DCM, and dried to afford valbenazine oxalate as an off-white solid. (1 g, purity: 99.7%, yield: 55%). The XRPD pattern of crystalline form A3 was shown in table 4.

TABLE 4

| 2theta | d spacing | Intensity % |
|---|---|---|
| 4.67 | 18.90 | 11.39 |
| 6.62 | 13.34 | 100.00 |
| 7.01 | 12.61 | 80.20 |
| 7.33 | 12.07 | 66.93 |
| 9.50 | 9.31 | 39.45 |
| 11.50 | 7.69 | 13.31 |
| 11.94 | 7.41 | 29.01 |
| 13.52 | 6.55 | 26.57 |
| 14.26 | 6.21 | 14.62 |
| 17.30 | 5.13 | 35.72 |
| 17.67 | 5.02 | 13.92 |
| 17.98 | 4.93 | 34.70 |
| 18.55 | 4.78 | 11.21 |
| 20.23 | 4.39 | 32.13 |
| 21.03 | 4.22 | 41.29 |
| 21.63 | 4.11 | 13.36 |

### Example 5

### Preparation of Compound I Oxalate Crystalline Form A4

A solution of compound I (5.2 g, HPLC purity: 97.4%) in DCM (50 mL) was concentrated to about 15 mL under vacuum. After solvent exchange to acetonitrile (MeCN), the solution (total volume about 45 mL) was heated to 40-50° C., followed by drop-wise addition of a solution of anhydrous oxalic acid in MeCN (1.0 eq anhydrous oxalic acid in 3 vol MeCN) at 40-50° C. After being stirred and held for 1-2 h at 40-50° C., the mixture was cooled to 20-30° C. and stirred for 17 h. The resulting suspension was filtered. The cake was washed with MeCN, and dried to afford valbenazine oxalate as an off-white solid. (5.27 g, purity: 99.6%, yield: 83.4%). The XRPD pattern of crystalline form A4 was shown in table 5.

TABLE 5

| 2theta | d spacing | Intensity % |
|---|---|---|
| 3.83 | 23.05 | 12.77 |
| 5.27 | 16.78 | 100.00 |
| 8.30 | 10.65 | 43.86 |
| 8.65 | 10.23 | 60.77 |
| 11.05 | 8.01 | 11.23 |
| 11.83 | 7.48 | 30.10 |
| 12.45 | 7.11 | 41.45 |
| 13.61 | 6.50 | 37.43 |
| 14.08 | 6.29 | 33.31 |
| 16.08 | 5.51 | 14.05 |
| 17.03 | 5.21 | 47.68 |
| 17.92 | 4.95 | 13.09 |
| 18.67 | 4.75 | 23.08 |
| 19.11 | 4.64 | 18.76 |
| 19.58 | 4.53 | 20.54 |
| 19.98 | 4.44 | 42.56 |

### Example 6

### Preparation of Compound I Oxalate Crystalline Form A4

A solution of compound I (5.69 g, HPLC purity: 95.63%) in DCM (90 mL) was concentrated under vacuum. MeCN (130 mL) was added. The MeCN solution was heated to 40-50° C., followed by drop-wise addition of a solution of anhydrous oxalic acid in MeCN (1.0 eq anhydrous oxalic acid in 3 vol MeCN) at 40-50° C. After being stirred and held for 0.5 h at 40-50° C., the mixture was cooled to 20-30° C. and stirred for 17 h. The resulting suspension was filtered. The cake was washed with MeCN, and dried to afford valbenazine oxalate as an off-white solid. (6.09 g, purity: 99.66%, yield: 88.13%). The XRPD pattern of crystalline form A4 was shown in table 6.

TABLE 6

| 2theta | d spacing | Intensity % |
|---|---|---|
| 4.00 | 22.09 | 14.81 |
| 5.50 | 16.07 | 100.00 |
| 8.54 | 10.35 | 40.14 |
| 8.80 | 10.05 | 45.29 |
| 11.99 | 7.38 | 22.23 |
| 12.65 | 7.00 | 28.59 |
| 13.82 | 6.41 | 29.09 |
| 14.26 | 6.21 | 22.67 |
| 16.27 | 5.45 | 13.04 |
| 17.27 | 5.13 | 38.76 |
| 18.09 | 4.90 | 13.10 |
| 18.77 | 4.73 | 14.99 |
| 19.25 | 4.61 | 18.73 |

### Example 7

### Preparation of Compound I Oxalate Crystalline Form A5

A solution of compound I (3.8 g, HPLC purity: 93.63%) in DCM (70 mL) was concentrated under vacuum. MeCN (100 mL) was added. The MeCN solution was heated to 40-50° C., followed by drop-wise addition of a solution of anhydrous oxalic acid (1.0 eq anhydrous oxalic acid in 4 vol MeCN) in MeCN at 40-50° C. After being stirred and held for 1.5 h at 40-50° C., the mixture was cooled to 20-30° C. and stirred for 20 h. The resulting suspension was filtered. The cake was washed with MeCN, and dried to afford valbenazine oxalate as an off-white solid. (3.36 g, purity: 99.52%, yield: 72.7%). The XRPD pattern of crystalline form A5 was shown in table 7.

TABLE 7

| 2theta | d spacing | Intensity % |
|---|---|---|
| 3.39 | 26.06 | 44.26 |
| 6.05 | 14.60 | 25.52 |
| 7.04 | 12.55 | 100.00 |
| 9.40 | 9.41 | 11.10 |
| 10.40 | 8.51 | 8.24 |
| 10.74 | 8.24 | 15.68 |
| 15.77 | 5.62 | 15.29 |
| 19.28 | 4.60 | 20.33 |
| 20.47 | 4.34 | 13.33 |
| 20.76 | 4.28 | 10.73 |

### Example 8

### Preparation of Compound I Hydrobromide

A solution of compound I (6.8 g, HPLC purity: 98.02%) in DCM (120 mL) was concentrated to 1-2 vol under vacuum. The solvent was exchanged to IPAc. The IPAc solution (total volume about 75 mL) was cooled to 10-20° C., followed by drop wise addition of a solution of 33 w/w % HBr in AcOH (2.8 g, 2.1 eq). The mixture was heated to 20-30° C. After being stirred and held for 2 h at 20-30° C. The resulting suspension was filtered. The cake was washed with IPAc, and dried to afford valbenazine hydrobromide as a light-yellow solid. (3 g, purity: 95.73%, yield: 31.8%). ¹H-NMR (DMSO-d₆, 400 MHz) δ: 6.81 (2H, 2s, ArH), 5.05 (1H, td, J=10.4 Hz, 4.0 Hz), 4.46 (1H, m), 3.95 (1H, d, J=5.2 Hz), 3.75 (6H, —OCH₃), 3.61 (1H, m), 3.54 (1H, m), 3.23 (2H, m), 3.06 (1H, m), 2.89 (2H, m), 2.39 (1H, m), 2.22 (1H, m), 1.90 (1H, m), 1.66 (1H, m), 1.33 (1H, m), 1.10 (1H, m), 1.03 (6H, dd, J=7.2 Hz, 10.0 Hz, -iPr), 0.91 (6H, dd, J=3.6 Hz, 6.4 Hz, -iPr).

### Example 9

### Preparation of Compound I (−)-Di-p-Toluoyl-L-Tartrate

A solution of compound I (379.3 mg, HPLC purity: 97.5%) in DCM (11 mL) was added (−)-Di-p-toluoyl-L-tartaric acid (1.0 eq). The solvent was exchanged to IPAc. The resulting suspension (about 45 mL) was heated to 50-60° C. After being stirred and held for 3 h, the mixture was cooled to 20-30° C. The suspension was filtered. The cake was washed with IPAc, and dried to afford valbenazine (−)-Di-p-toluoyl-L-tartrate as a light-yellow solid. (600 mg, purity: 99.2%, yield: 82%). ¹H-NMR (DMSO-d₆, 400 MHz) δ: 7.86 (4H, d, J=8.0 Hz, ArH), 7.31 (4H, d, J=8.0 Hz, ArH), 6.64 (2H, 2s, ArH), 5.64 (2H, s), 4.73 (brs, active hydrogen), 4.68 (1H, td, J=4.8 Hz, 10.8 Hz), 3.72 (1H, d, J=4.8 Hz), 3.69 (6H, —OCH₃), 3.29 (1H, brd, J=11.6 Hz), 3.07 (1H, dd, J=4.0 Hz, 12.0 Hz), 3.01 (1H, m), 2.89 (1H, m), 2.56 (1H, m), 2.45 (1H, m), 2.36 (6H, s, ArCH3), 2.09 (2H, m), 1.86 (1H, m), 1.41 (1H, m), 1.23 (1H, m), 0.95 (1H, m), 0.90 (6H, dd, J=5.6 Hz, 6.8 Hz, -iPr), 0.85 (6H, dd, J=6.4 Hz, 10.8 Hz, -iPr).

The XRPD pattern of crystalline form C was shown in table 8.

TABLE 8

| 2theta | d spacing | Intensity % |
|---|---|---|
| 5.41 | 16.35 | 41.66 |
| 6.63 | 13.32 | 100.00 |
| 7.90 | 11.20 | 11.96 |
| 10.85 | 8.15 | 5.40 |
| 13.30 | 6.66 | 21.42 |
| 13.99 | 6.33 | 49.46 |
| 15.87 | 5.59 | 9.76 |
| 16.61 | 5.34 | 16.76 |
| 18.75 | 4.73 | 13.20 |
| 20.00 | 4.44 | 6.50 |
| 22.06 | 4.03 | 7.95 |

### Example 10

### Preparation of Compound I D-Tartrate

A solution of compound I (1.52 g, HPLC: 97.49%) in DCM (15 g) was added D-tartaric acid (1.0 eq). The solvent was exchanged to MeCN. The resulting suspension (about 25 mL) was heated to 40-50° C. After being stirred and held for 3 h, the mixture was cooled to 20-30° C. and held for about 16 h. The suspension was filtered. The cake was washed with MeCN, and dried to afford valbenazine D-tartrate as an off-white solid. (1.86 g, purity: 98.59%, yield: 90%). ¹H-NMR (DMSO-d₆, 400 MHz) δ: 6.66 (2H, 2s, ArH), 5.82 (brs, active hydrogen), 4.72 (1H, td, J=4.8 Hz, 10.8 Hz), 4.07 (2H, s), 3.70 (6H, —OCH₃), 3.66 (1H, d, J=4.8 Hz), 3.25 (1H, brd, J=11.2 Hz), 3.06 (1H, m), 2.99 (1H, m), 2.90 (1H, m), 2.55 (2H, m), 2.41 (1H, m), 2.11 (2H, m), 1.85 (1H, m), 1.63 (1H, m), 1.41 (1H, m), 1.27 (1H, m), 1.02 (1H, m), 0.97 (6H, dd, J=2.8 Hz, 7.2 Hz, -iPr), 0.87 (6H, dd, J=6.8 Hz, 10.8 Hz, -iPr).

The XRPD pattern of crystalline form D was shown in table 9.

TABLE 9

| 2theta | d spacing | Intensity % |
|---|---|---|
| 6.67 | 13.25 | 100.00 |
| 7.78 | 11.37 | 27.17 |
| 11.22 | 7.89 | 25.53 |
| 15.31 | 5.79 | 18.14 |
| 16.27 | 5.45 | 21.12 |
| 18.19 | 4.88 | 36.31 |
| 18.56 | 4.78 | 15.58 |
| 19.70 | 4.51 | 18.01 |
| 20.46 | 4.34 | 21.55 |
| 21.93 | 4.05 | 22.35 |

### Example 11

### Preparation of Compound I L-(+)-Tartrate

A solution of compound I (1.52 g, HPLC: 97.49%) in DCM (15 g) was added L-(+)-tartaric acid (1.0 eq). The solvent was exchanged to MeCN. The resulting suspension (about 25 mL) was heated to 40-50° C. After being stirred and held for 3 h, the mixture was cooled to 20-30° C. and held for about 16 h. The suspension was filtered. The cake was washed with MeCN, and dried to afford valbenazine L-tartrate as an off-white solid. (1.7 g, purity: 99.44%, yield: 82.3%). ¹H-NMR (DMSO-d₆, 400 MHz) δ: 6.66 (2H, 2s, ArH), 5.82 (brs, active hydrogen), 4.71 (1H, td, J=4.4 Hz, 10.4 Hz), 4.06 (2H, s), 3.70 (6H, —OCH₃), 3.67 (1H, d, J=4.8 Hz), 3.25 (1H, brd, J=11.2 Hz), 3.06 (1H, m), 2.98 (1H, m), 2.90 (1H, m), 2.55 (2H, m), 2.41 (1H, m), 2.11 (2H, m), 1.85 (1H, m), 1.64 (1H, m), 1.40 (1H, m), 1.26 (1H, m), 1.02 (1H, m), 0.97 (6H, dd, J=2.4 Hz, 6.8 Hz, -iPr), 0.87 (6H, dd, J=6.4 Hz, 10.8 Hz, -iPr).

The XRPD pattern of crystalline form B was shown in table 10.

TABLE 10

| 2theta | d spacing | Intensity % |
|---|---|---|
| 9.78 | 9.05 | 93.46 |
| 10.03 | 8.82 | 81.91 |
| 11.02 | 8.03 | 100.00 |
| 12.10 | 7.32 | 41.31 |
| 14.24 | 6.22 | 97.47 |
| 14.86 | 5.96 | 52.35 |
| 16.60 | 5.34 | 42.97 |
| 18.60 | 477 | 49.06 |
| 20.04 | 4.43 | 45.21 |
| 21.45 | 4.14 | 43.69 |

### Contrast Example 12

### Preparation of Compound I Hydrochloride

A solution of compound I (37.9 g, HPLC: 97.49%) in DCM was concentrated under vacuum. After solvent exchange to MeCN, the solution (total volume about 470 mL) was added drop-wise a solution of HCl-IPA (3.7 M, 52 mL). EtOAc (110 mL) was added. The suspension (about 630 mL) was heated to 45-55° C. After being added a second portion of EtOAc (about 770 mL), the mixture was heated to reflux for 1 h. After being cooled to 20-30° C., the resulting suspension was filtered. The cake was washed with EtOAc, and dried to afford valbenazine hydrochloride as an off-white solid. (36.06 g, purity: 93.03%, yield: 81.03%).

### Example 13

### Preparation of Compound I Crystalline Form X

A solution of compound I (15.8 g, HPLC: 99.6%) in DCM (120 mL) was concentrated under vacuum. After solvent exchange to n-hexane, the suspension (about 80 mL) was stirred and held for 2 h at 25-35° C. The resulting suspension was filtered. The cake was washed with n-hexane, and dried to afford valbenazine free base as an off-white solid. (13.14 g, purity: 99.69%, yield: 83.2%). The XRPD pattern of crystalline form X was shown in table 11.

TABLE 11

| 2theta | d spacing | Intensity % |
|---|---|---|
| 5.92 | 14.94 | 58.90 |
| 6.67 | 13.26 | 56.94 |
| 8.30 | 10.66 | 22.70 |
| 9.85 | 8.98 | 100.00 |
| 11.94 | 7.41 | 8.99 |
| 13.43 | 6.59 | 29.09 |
| 14.12 | 6.27 | 74.76 |
| 16.31 | 5.44 | 7.40 |
| 16.72 | 5.30 | 30.39 |
| 17.99 | 4.93 | 56.18 |
| 19.80 | 4.48 | 7.51 |
| 20.15 | 4.41 | 53.23 |
| 22.16 | 4.01 | 5.43 |
| 23.83 | 3.73 | 5.47 |

### Example 14

### Stability Data of Form A1 of Valbenazine Oxalate

Stability HPLC data summary of Form A1 of valbenazine oxalate
Store Package The Form A1 of valbenazine oxalate was packaged
in double PE bags. Sealed in a laminated
aluminum foil bag.
Storage conditions 2~8° C.

| | | Time | | | | | | | |
|---|---|---|---|---|---|---|---|---|---|
| | | 0 months | | 0.5 months | | 1 months | | 3 months | |
| | | Appearance | | | | | | | |
| | | White | | | | | | | |
| | | solid | | White solid | | White solid | | White solid | |
| Assay | | 100.1% | | 100.8% | | 101.0% | | 100.33% | |
| (Anhydrous) | | | | | | | | | |
| HPLC | Valben- | 99.81% | | 99.84% | | 99.84% | | 99.78% | |
| | azine | | | | | | | | |
| | Imp-1 | 0.03% | LSI | 0.03% | LSI | 0.03% | LSI | 0.04% | LSI |
| | Imp-2 | 0.01% | | ND | | 0.01% | | 0.02% | |
| | Imp-3 | 0.02% | | 0.03% | | 0.02% | | 0.04% | |
| | Imp-4 | 0.02% | | ND | | 0.01% | | 0.03% | |
| | Imp-5 | 0.01% | | 0.01% | | 0.01% | | 0.02% | |
| | Imp-6 | ND | | ND | | ND | | ND | |
| | Imp-7 | 0.01% | | 0.02% | | 0.01% | | 0.01% | |
| | Imp-8 | ND | | ND | | ND | | ND | |
| | Imp-9 | ND | | ND | | ND | | ND | |
| Chiral | Valben- | 99.93% | | 99.93% | | 99.95% | | 99.91% | |
| HPLC | azine | | | | | | | | |
| | Chiral | 0.01% | | 0.01% | | 0.01% | | 0.01% | |
| | imp-1 | | | | | | | | |
| | Chiral | 0.04% | | 0.04% | | 0.04% | | 0.07% | |
| | imp-2 | | | | | | | | |

| | | | | |
|---|---|---|---|---|
| Chiral | 0.03% | 0.03% | ND | 0.02% |
| imp-3 | | | | |

| | | | | |
|---|---|---|---|---|
| Moisture | 0.12% | 0.2% | 0.19% | 0.4% |

Stability XRPD data summary of Form A1 of valbenazine oxalate

| | | | Final solid |
|---|---|---|---|
| Initial solid Form | condition | time | Form |
| A1 | 2-8° C. | 3 months | A1 |

### Example 15

### Purification Capability Data Valbenazine Oxalate Vs Valbenazine Tosylate and Hydrochloride

| | Purity, Area % | | | | Chiral purity, Area % | | | |
|---|---|---|---|---|---|---|---|---|
| | Form | Form | | | Form | Form | | |
| | A1 | A1 | | Valbenazine | A1 | Al | | |
| | Batch | Batch | Valbenazine | Batch | Batch | Batch | Valbenazine | Valbenazine |
| Entry | 1 ¹ | 2 ¹ | hydrochloride ² | tosylate ³ | 1 ¹ | 2 ¹ | hydrochloride ² | tosylate ³ |
| Crude | 97.5% | 97.4% | 97.45% | 97.0% | 97.4% | 98.0% | 98.98% | 98.98% |
| valbenazine | | | | | | | | |
| Valbenazine | 99.4% | 99.6% | 93.03% | 97.4% | 99.8% | 99.9% | 98.85% | 98.85% |
| salt | | | | | | | | |

¹ Prepared following the process in example 1;
² Prepared following the process in WO2017075340 example 14;
³ Prepared following the process in WO2017075340 example 2.

1. An oxalate salt of L-Valine, (2R,3R,11bR)-1,3,4,6,7,11b-hexahydro-9,10-dimethoxy-3-(2-methylpropyl)-2H-benzo[a]quinolizin-2-yl ester in a crystalline form selected from the group consisting of Form A1, Form A2, Form A3, Form A4 and Form A5.

2. (canceled)

3. (canceled)

4. The oxalate salt of claim 1, wherein the crystalline form is Form A1 having an X-ray powder diffraction pattern comprising peaks at 5.4, 7.1 and 13.4 degrees two theta±0.2 theta using λ at 1.54 A.

5. The oxalate salt of claim 1, wherein the crystalline form is Form A2 having an X-ray powder diffraction pattern comprising peaks at 15.1, 20.2 and 20.6 degrees two theta±0.2 theta using λ at 1.54 A.

6. The oxalate salt of claim 1, wherein the crystalline form is Form A3 having an X-ray powder diffraction pattern comprising peaks at 6.7, 7.1 and 9.5 degrees two theta±0.2 theta using λ at 1.54 A.

7. The oxalate salt of claim 1, wherein the crystalline form is Form A4 having an X-ray powder diffraction pattern comprising peaks at 5.5, 8.8 and 12.7 degrees two theta±0.2 theta using λ at 1.54 A.

8. The oxalate salt of claim 1, wherein the crystalline form is Form A5 having an X-ray powder diffraction pattern comprising peaks at 3.4, 6.1 and 7.0, degrees two theta±0.2 theta using λ at 1.54 A.

9. A hydrobromide salt of L-Valine, (2R,3R,11bR)-1,3,4,6,7,11b-hexahydro-9,10-dimethoxy-3-(2-methylpropyl)-2H-benzo[a]quinolizin-2-yl ester in amorphous form.

10. (canceled)

11. A L-tartrate salt of L-Valine, (2R,3R,11bR)-1,3,4,6,7,11b-hexahydro-9,10-dimethoxy-3-(2-methylpropyl)-2H-benzo[a]quinolizin-2-yl ester in a crystalline form.

12. (canceled)

13. The L-tartrate salt of claim 11, wherein the crystalline form is Form B.

14. The L-tartrate salt of claim 13, wherein Form B has an X-ray powder diffraction pattern comprising peaks at 9.8, 11.0 and 14.2 degrees two theta±0.2 theta using λ at 1.54 A.

15. A Di-p-toluoyl-L-tartrate salt of L-Valine, (2R,3R,11bR)-1,3,4,6,7,11b-hexahydro-9,10-dimethoxy-3-(2-methylpropyl)-2H-benzo[a]quinolizin-2-yl ester.

16. The Di-p-toluoyl-L-tartrate salt of claim 15, wherein the salt is in a crystalline form.

17. The Di-p-toluoyl-L-tartrate salt of claim 16, wherein the crystalline form is Form C.

18. The Di-p-toluoyl-L-tartrate salt of claim 17, wherein Form C has an X-ray powder diffraction pattern comprising peaks at 5.4, 6.6 and 14.0 degrees two theta±0.2 theta using λ at 1.54 A.

19. A D-tartrate salt of L-Valine, (2R,3R,11bR)-1,3,4,6,7,11b-hexahydro-9,10-dimethoxy-3-(2-methylpropyl)-2H-benzo[a]quinolizin-2-yl ester.

20. The D-tartrate salt of claim 19, wherein the salt is in a crystalline form.

21. The D-tartrate salt of claim 20, wherein the crystalline form is Form D.

22. The D-tartrate salt of claim 21, wherein Form D has an X-ray powder diffraction pattern comprising peaks at 6.7, 7.8 and 18.2 degrees two theta±0.2 theta using λ at 1.54 A.
